(19) United States
(12) Patent Application Publication
(10) Pub. No.: US 2017/0333515 A1
(43) Pub. Date: Nov. 23, 2017

(54) COMPOSITIONS AND METHODS FOR ENHANCING THE METABOLIC ACTIVITY OR STABILITY OF CURCUMIN

(71) Applicant: Eric H. Kuhrts, Fairfield, CA (US)

(72) Inventor: Eric H. Kuhrts, Fairfield, CA (US)

(21) Appl. No.: 15/348,417

(22) Filed: Nov. 10, 2016

Related U.S. Application Data
(60) Provisional application No. 62/253,263, filed on Nov. 10, 2015.

(51) Int. Cl. A61K 36/9066; A61K 47/40; A61K 47/14; A61K 47/69; A23L 27/10; A61K 31/05; A61K 9/14; A61K 9/08; A61K 31/121; A61K 47/10

(52) U.S. Cl. CPC A61K 36/9066; A61K 31/121; A61K 47/40; A61K 47/14; A61K 47/10; A23L 27/10; A61K 31/05; A61K 9/148; A61K 9/08; A61K 47/6937; A61K 2300/00

(57) ABSTRACT

A metabolic enhancing pharmaceutical composition can include xanthohumol and curcumin, and these components can be in extract form, for example. In another example, a method of enhancing the metabolic stability of a curcumin extract in the body can include combining a curcumin extract with an extract of xanthohumol from hops.

### BACKGROUND

It is well known that impaired neurotransmission, resulting from low neurotransmitter levels and/or decreased neurotransmitter receptor affinity, is related to mental disease, such as depression, generalized anxiety disorder (GAD), and increased susceptibility to stress and cognitive dysfunction. Compounds that increase neurotransmitter levels in the brain and thus enhance their transmission can exhibit antidepressant properties and exert beneficial effects on a variety of other mental disorders. The main neurotransmitters are serotonin, dopamine, noradrenaline (norepinephrine), acetylcholine, glutamate and gamma-aminobutyric acid (GABA). Neurotransmitters of particular relevance to mood-related disorders include serotonin, noradrenaline, and dopamine, while glutamate and acetylcholine neurotransmission are involved in cognitive function. Enhanced or prolonged neurotransmission is achieved by increasing the concentration of the neurotransmitter in the synaptic cleft, through inhibition of re-uptake into the pre-synaptic nerve ending, or by preventing neurotransmitter catabolism by inhibition of degrading enzymes, such as monoamine oxidase (MAO)-A and -B. Also of interest are the neuropeptides, such as calcitonin gene-related peptide (CGRP).

Commonly prescribed tricyclic antidepressants (TCA), such as imipramine, amitriptyline, and clomipramine, act by inhibiting the re-uptake of serotonin and noradrenaline. These drugs are widely regarded as among the most effective antidepressants available, but unfortunately have a number of disadvantages because they additionally interact with muscarinic, acetylcholine, histamine, and serotonin receptors. Side effects resulting from the use of these drugs include dry mouth, blurred vision, constipation and urinary retention, in addition to postural hypotension. Also, TCAs are not safe when taken in overdose, frequently showing acute cardiotoxicity and other possible side-effects.

Another class of antidepressant drugs are selective serotonin re-uptake inhibitors (SSRI). This class of drugs includes fluoxetine, paroxetine, sertraline, citalopram and fluvoxamine, and act by blocking the serotonin transporter (SERT), a high affinity sodium chloride-dependent neurotransmitter transporter that terminates serotonergic neurotransmission by re-uptake of serotonin. These drugs have been proven effective in the treatment of depression and anxiety as TCAs, and are usually better tolerated. These medications are typically started at low dosages and are increased until they reach a therapeutic level. A common side effect is nausea. Other possible side effects include decreased appetite, dry mouth, sweating, infection, constipation, tremor, yawning, sleepiness and sexual dysfunction.

In addition, compounds that prevent the catabolism of neurotransmitters more broadly by inhibiting MAOs-A and -B exhibit antidepressant effects. MAOs work by catalyzing the oxidation of amine group-containing neurotransmitters such as serotonin, noradrenaline and dopamine.

There is a need for therapeutic compounds for the treatment or prevention of mental diseases and/or disorders which do not show the negative side effects of known antidepressants. Patients with mood disorders are interested in alternative therapies which could minimize the side effects associated with high doses of drugs and yield additional clinical benefits. Severe depression is a long-lasting and recurring disease, which is usually poorly diagnosed. Mild depression is a much more common problem in modern society, and many more patients suffer from mild or moderately severe depression. Thus, there is an increasing interest in the development of therapeutic compounds, as well as pharmaceutical and/or dietary compositions, which may be used to treat or prevent mental diseases/disorders such as depression and dysthymia, in people at risk, to stabilize mood and achieve emotional balance.

Mood disorders, emotional imbalance, and occupational stress can lead to sleep disorders, insomnia, low sleep quality and general disturbances in circadian rhythms (so-called biorhythms), and such conditions can be chronic and persistent in nature. Also, dysregulation of circadian rhythms induced by long-haul flights (jet-lag) and shift-work can cause similar symptoms and distress. Therefore, treatment with therapeutic supplementation to maintain a normal circadian rhythm and/or to alleviate and prevent symptoms associated with a disturbed circadian rhythm, such as impairment of cognitive function and memory and mental and physical fatigue, and resulting in improving the overall quality of life and benefiting from improved social integration, would be most desirable.

Various natural botanical compounds that have been investigated for depression, anxiety, and insomnia. Among these compounds can be found extracts from borage, lavender, ginseng, mimosa, roseroot, saffron, St. John's wort, California poppy, chamomile, ginkgo, gotu cola, kava, lemonbalm, passion flower, skullcap, withania, and curcumin extracted from turmeric. Over the last few years, one of the most promising compounds to emerge from this group has been curcumin. A number of double blind, placebo controlled human clinical studies have been conducted that have demonstrated that curcumin, in the right formulation and potency, can help to alleviate mild depression, and in fact may be equally effective as some of the pharmaceuticals that are prescribed for this purpose.

However, curcumin, being a lipophilic compound, suffers from poor bioavailability or absorption.

### SUMMARY

The present disclosure is drawn a metabolic enhancing pharmaceutical composition comprising xanthohumol and curcumin. In one example, the curcumin is from a curcumin extract and the xanthohumol is from a xanthohumol extract from hops. In another example, the xanthohumol is present at a concentration from about 0.01% to 50% by weight, and the curcumin is present at a concentration of from 0.01% to 10% by weight. The formulation can further include an emulsifier, ethanol, d-alpha tocopheryl polyethylene glycol succinate, glycerin, cycoldextrin, phosphatidylcholine, PEGylated nanospheres, PLGA nanospheres, or nanoparticles. In another example, there can be a non-ionic surfactant included. In still another example, the xanthohumol and curcumin can be present as extracts, and the extracts to non-ionic surfactant weight ratio is from about 1:5 to about 1:200.

In another example, a method of enhancing the metabolic activity or stability of a curcumin extract can include preparing a formulation by combining the curcumin extract with an extract of xanthohumol from hops. The formulation can be administered with enhanced metabolic stability or activity in a subject compared to the administration of the curcumin extract with the extract of xanthohumol, for example. The curcumin and xanthohumol can be formulated with a solubility enhancing agent, such as an emulsifier, ethanol, non-ionic surfactant, d-alpha tocopheryl polyethylene glycol succinate, glycerin, cycoldextrin, phosphatidylcholine, PEGylated nanospheres, PLGA nanospheres, or nanoparticles. The formulation can be administered in the form of a water soluble gel or concentrate. The formulation can include a solid or liquid carrier suitable to form a consumable formulation. When a solid carrier, it can include magnesium carbonate, magnesium stearate, talc, sugar, lactose, pectin, dextrin, starch, gelatin, tragacanth, a low melting wax, cocoa butter, vegetable oil, ethanol, sucrose, mannitol, sorbitol, cellulose, gums, gelatin, collagen, or combination thereof. When a liquid carrier, the consumable formulation can be in the form of a beverage. Other example consumable formulations can include beverages, food, feed, dairy products, yoghurts, fortified food, enhanced waters, cereal bars, bakery items, cakes, cookies, dietary supplements, tablets, pills, granules, dragees, capsules, effervescent formulations, non-alcoholic or alcoholic drinks, soft drinks, sport drinks, fruit juices, teas, milk-based drinks, liquid foods, soups, liquid dairy products, or any combination thereof.

### DETAILED DESCRIPTION

In describing and claiming the present invention, the following terminology will be used.

The singular forms “a,” “an,” and “the” include plural referents unless the context clearly dictates otherwise. Thus, for example, reference to “a non-ionic surfactant” includes reference to one or more of such compounds.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

In the context of the present disclosure, the term “disorder” also encompasses diseases.

As used herein, a “prenylflavonoid,” or prenylaflavonoid, refers to a prenylated compound having a substituted or unsubstituted phenol attached to a phenyl via a C3 alkylene substituted with an oxo group. The C3 alkylene may be present in a linear chain arrangement (e.g. a chalcone) or joined with other atoms to form a substituted or unsubstituted ring (e.g. a flavanone). Prenylflavonoids may be derived from natural sources (e.g. hops), or synthesized chemically. A “prenylated” compound refers to those compounds with an attached —CH₂—CH═C(CH₃)₂ group (e.g. geranylated compounds), optionally hydroxylated prenyl tautomers (e.g. —CH₂—CH—C(CH₃)═CH₂, or —CH₂—C(OH)—C(CH₃)═CH₂), and optionally hydroxylated circularized prenyl derivatives having the formula below:

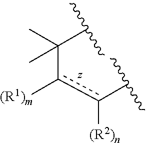

In this formula, the dashed bond z represents a double bond or a single bond. R¹ and R² are independently hydrogen or OH. The symbol [symbol] represents the point of attachment to the remainder of the prenylated compounds.

As used herein, the term “extracted prenylflavonoid” refers to a xanthohumol hops extract that has been extracted by any number of extraction methods including ethanol extractions, supercritical carbon dioxide extractions, or the like. The xanthohumol content, after extraction, will typically be at least 3% wt % pure, but can be 5 wt %, 10 wt %, 30 wt %, 40%, 50%, 80% or even 99 wt % pure.

Hops (Humulus lupulis L.) has been used for centuries as a bittering agent in the brewing of beer. Hops contains alpha acids such as humulone, co-humuone, ad-humulone, and beta acids such as lupulone and co-lupulone. Hops also contains many flavonoids, such as xanthohumol, isoxanthohumol, desmethylxanthohumol, 8-prenylnaringenin, and 6-prenylnaringenin. Xanthohumol is a yellow-orange substance with a melting point of 172 degrees C. A typical ethanol extract of hops yields about 3 mg./g (3%) of xanthohumol out of a total flavonoid content of 3.46 mg./g. Dried hop contains about 0.2 to 1.0% by weight xanthohumol. Xanthohumol, molecular weight is 354.40, CAS 6754-58-1, C21H22O5

As used herein, the term curcumin means 1,7-Bis(4-hydroxy-3-methoxyphenyl)-1,6-heptadiene-3,5-dione, the principle polyphenol (biphenyl) compound extracted from the turmeric root. Turmeric is a spice that consists of the dried powder derived from the rhizome of Curcuma longa.

Turmeric has been used since ancient times in Indian traditional medicine (Ayurveda) and Traditional Chinese Medicine (TCM). Turmeric is also a popular spice, and is part of the seasoning curry. Turmeric has been in use in Asian medicine for over 3,000 years. Scientific research over the last 10-15 years has indicated that curcumin has significant potential for treating numerous health problems such as anti-inflammatory, anti-oxidative, cancer chemoprevention, anti-cancer, wound healing, antimicrobial, and more recently as an anti-depressant.

The rhizome of the plant Curcuma longa, which is commonly known as turmeric in the English language, is also known as ukon in Japanese, and haldi in Hindi.

A “non-ionic surfactant,” as used herein, is a surface active agent that tends to be non-ionized (i.e. uncharged) in neutral solutions (e.g. neutral aqueous solutions).

As used herein, “increase in metabolic stability” means a change in the metabolic profile of a compound when combined with another compound, and tested by determining the percent remaining of the test compound incubated with human, rat, dog, primate, or mouse liver microsomes in the presence of NADPH. The stability of one compound is tested in liver microsomes in the absence and presence of the other compound. An increase in stability is indicated by a change (increase) in the half-life and the modulation of the intrinsic clearance of the compound.

A “transparent” or “clear” water soluble formulation, as disclosed herein, refers to a formulation that can be clearly seen through with the naked eye and is optionally colored.

In the present disclosure, unless the context dictates otherwise, all percentages are based on weight.

Concentrations, amounts, solubility, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a numerical range of about 1 to about 4.5 should be interpreted to include not only the explicitly recited limits of 1 to about 4.5, but also to include individual numerals such as 2, 3, 4, and sub-ranges such as 1 to 3, 2 to 4, etc. The same principle applies to ranges reciting only one numerical value, such as “less than about 4.5,” which should be interpreted to include all of the above-recited values and ranges. Further, such an interpretation should apply regardless of the breadth of the range or the characteristic being described.

With this in mind, in the search for alternative mechanisms to treat mood disorders, it has been found that xanthohumol, a prenylflavonoid derived from the flowers of the hops plant, Humulus lupulus L., when combined with curcumin, extracted from turmeric, has great potential in the treatment of emotional imbalance, depression, mood disorders, and various other mental disturbances. These affective emotional states also relate to anti-social behavior and relationships between people such as married couples, family members, and others in a societal context.

Furthermore, there is an increasing interest in the development of therapeutic compounds that may be used to improve focus, learning, memory and alertness, in both elderly and young people, individuals who need especially high memory and attention in their daily work, including students, construction workers, drivers, pilots, physicians, salespeople, executives, housewives, “high performance professionals” and people who are under mental or daily stress as well as persons who are prone to psychiatric instability and dramatic mood swings. A therapeutic product that can provide a balanced mental state would contribute to a more productive life for a large percentage of people who live a high stress, high performance life.

Thus, a compound or nutraceutical composition which may increase the beneficial properties of curcumin, thereby enabling improvements in learning, memory and alertness, enabling improved mood, would be desirable. Some or all of these advantages may be possible using the formulations of the present disclosure.

In accordance with this, a composition and method for the treatment of mood disorders can comprise administering an extracted hops xanthohumol prenylflavonoid/curcumin polyphenol combination to a subject. The combination of the two ingredients has been found to be more effective than either ingredient alone. Furthermore, it has now been discovered that the combination of xanthohumol from hops with curcumin extracted from turmeric, improves the stability and increases the half-life of curcumin, which is an unexpected discovery. Additional ingredients or formulations designed to effect the bioavailability of either substance can also be included, such as non-ionic surfactant, d-alpha tocopheryl polyethylene glycol succinate (1000 succinate), glycerin, cycoldextrin, phosphatidylcholine, PEGylated compounds, PLGA nanospheres, nanoparticles, and/or other additives or excipients, some of which may be incorporated to increase the absorption of the curcumin or xanthohumol. In one example, a stable, water-soluble pharmaceutical gel composition of the prenylflavonoid xanthohumol with the polyphenol curcumin is disclosed that can be prepared and administered as described herein. In this method, a water-soluble non-ionic surfactant is heated in a container to a temperature of about 90° F. to about 200° F. while mixing the non-ionic surfactant until a clear non-ionic surfactant is formed. An extracted prenylflavonoid (xanthohumol) or polyphenol (curcumin) is then added to the clear non-ionic surfactant and mixed until a clear non-ionic surfactant-curcumin/xanthohumol combination is formed so as to constitute from about 20 wt % to 50 wt % surfactant, and from 0.01 wt % to 10 wt % prenylflavonoid (xanthohumol) or polyphenol (curcumin), wherein the prenylflavonoid or polyphenol is sufficiently dispersed or dissolved in the surfactant so that a gel composition is formed containing no visible micelles or particles of prenylflavonoid/polyphenol. In administering this or other formulations to a subject, the resultant water-soluble pharmaceutical gel or concentrate can be administered, or it can be admixed with a liquid or solid carrier for administration.

The prenylflavonoid may be derived from a natural source, such as hops. Hops (Humulus lupulus L.) has been used for centuries as a bittering agent in the brewing of beer. Hops contain alpha acids such as humulone, co-humulone, ad-humulone, and beta acids such as lupulone and co-lupulone. Hops also contains many prenylflavonoids, such as xanthohumol, isoxanthohumol, desmethylxanthohumol, 8-prenylnaringenin, and 6-prenylnaringenin. Xanthohumol is a yellow-orange substance with a melting point of 172 degrees C. and a molecular weight of 354.4. A typical ethanol extract of hops yields about 3 mg/g (3%) of xanthohumol out of a total flavonoid content of 3.46 mg/g. Dried hop contains about 0.2 to 1.0% by weight xanthohumol. Xanthohumol can be extracted and purified to a concentration of greater than 1 wt %, in certain examples, to greater than 5 wt %, 20 wt %, 40 wt %, 80 wt %, or even 99 wt % pure. By extracting xanthohumol from hops, and formulating into a suitable food, supplement, beverage, or other medicinal dosage form with curcumin, an effective formulation for treating mood disorders or other health problems, and prolonging the half-life of curcumin in the body is possible.

Xanthohumol may be isolated from hops through purification, fractionation, or separation methods that are known to those skilled in the art. Ethanol may be used to extract higher levels of the prenylflavonoids from hops. The typical prenylflavonoid content of an ethanol extract of hops includes xanthohumol (3 mg/g), desmethylxanthohumol (0.34 mg/g), isoxanthohumol (0.052 mg/g), 6-prenylnaringenin (0.061 mg/g), and 8-prenylnaringenin 0.015 (mg/g). Supercritical carbon dioxide extractions tend to contain much lower levels, or non-existent levels of prenylflavonoids. In fact, these compounds are almost non-existent in standard CO₂ extracts because the prenylflavonoids are virtually insolvent on carbon dioxide. In the examples provided herein, a xanthohumol extract of purity of greater than 5 wt % has been used. It is noted herein that any method used to isolate prenylflavonoids are referred to herein as “extractions” or “extracted prenylflavonoids, regardless of how the isolation or concentration occurs.

Extracted prenylflavonoids that are useful as described herein can include prenylchalcones and/or prenylflavanones. In some embodiments, the prenylflavonoid is selected from xanthohumol, xanthogalenol, desmethylxanthohumol (2′,4′,6′,4-tetrahydrooxy-3-C-prenylchalcone), 2′,4′,6′,4-tetrahydrooxy-3′-C-geranylchalcone, dehydrocycloxanthohumol, dehydrocycloxanthohumol hydrate, 5′-prenylxanthohumol, tetrahydroxanthohumol, 4′-O-5′-C-diprenylxanthohumol, chalconaringenin, isoxanthohumol, 6-prenylnaringenin, 8-prenylnaringenin, 6,8-diprenylnaringenin, 4′,6′-dimethoxy-2′,4-dihydroxychalcone, 4′-O-methylxanthohumol, 6-geranylnaringenin, 8-geranylnaringenin, and metabolites and/or derivatives thereof. Prenylflavonoids of interest include xanthohumol, xanthohumol metabolites, and derivatives thereof, in extracted form. By extracting the prenylflavonoids and then using the extracted prenylflavonoids to prepare appropriate formulations, a more pure form of these ingredients can be prepared and used at an appropriate concentration for treating these conditions.

Extracted turmeric polyphenols such as curcumin can be extracted and purified using methods known to medicinal plant chemistry and pharmacology. Turmeric is a spice that consists of the dried powder derived from the rhizome of Curcuma longa. The turmeric extract used herein has been, purified using methods know to the art to contain a level of curcumin of 98-99%, as verified by HPLC.

In some embodiments, the hops prenylflavonoid xanthohumol can be present in the formulation at a concentration of at least 1%, 5%, 10%, 20%, 25%, 30%, 35%, 45%, 45%, or 50% by weight. In other embodiments the xanthohumol can be present in the pharmaceutical dosage form at a concentration from 0.01%, 0.1%, 1% to 80%, 5% to 50%, 10% to 35%, or 20% to 25% (by weight).

In some embodiments, the polyphenol curcumin can also be present in the formulation at a concentration of at least 1%, 5%, 10%, 20%, 25%, 30%, 35%, 45%, 45%, or 50% by weight. In other embodiments the curcumin can be present in the pharmaceutical dosage form at a concentration from 0.01%, 0.1%, 1% to 80%, 5% to 50%, 10% to 35%, or 20% to 25% (by weight).

If a water-soluble gel or concentrate is to be formed (and then mixed with water as described herein), useful non-ionic surfactants include, for example, non-ionic water soluble mono-, di-, and tri-glycerides; non-ionic water soluble mono- and di-fatty acid esters of polyethylene glycol; non-ionic water soluble sorbitan fatty acid esters (e.g. sorbitan monooleates such as SPAN 80 and TWEEN 20 (polyoxyethylene 20 sorbitan monooleate)); polyglycolyzed glycerides; non-ionic water soluble triblock copolymers (e.g. poly(ethyleneoxide)/poly-(propyleneoxide)/poly(ethyleneoxide) triblock copolymers such as POLOXAMER 406 (PLURONIC F-127), and derivatives thereof.

Examples of non-ionic water soluble mono-, di-, and tri-glycerides include propylene glycol dicarpylate/dicaprate (e.g. MIGLYOL 840), medium chain mono- and diglycerides (e.g. CAPMUL and IMWITOR 72), medium-chain triglycerides (e.g. caprylic and capric triglycerides such as LAVRAFAC, MIGLYOL 810 or 812, CRODAMOL GTCC-PN, and SOFTISON 378), long chain monoglycerides (e.g. glyceryl monooleates such as PECEOL, and glyceryl monolinoleates such as MAISINE), polyoxyl castor oil (e.g. macrogolglycerol ricinoleate, macrogolglycerol hydroxystearate, macrogol cetostearyl ether), and derivatives thereof.

Non-ionic water soluble mono- and di-fatty acid esters of polyethylene glycol include d-α-tocopheryl polyethylene glycol 1000 succinate (TPGS), polyethylene glycol 660 12-hydroxystearate (SOLUTOL HS 15), polyoxyl oleate and stearate (e.g. PEG 400 monostearate and PEG 1750 monostearate), and derivatives thereof.

Polyglycolyzed glycerides include polyoxyethylated oleic glycerides, polyoxyethylated linoleic glycerides, polyoxyethylated caprylic/capric glycerides, and derivatives thereof. Specific examples include LABRAFIL M-1944CS, LABRAFIL M-2125CS, LABRASOL, SOFTIGEN, and GELUCIRE.

In some embodiments, the non-ionic surfactant is a polyoxyl castor oil, or derivative thereof. The major component of the relatively hydrophobic portion is glycerol polyethylene glycol ricinoleate, and the major components of the relatively hydrophilic portion are polyethylene glycols and glycerol ethoxylates. Macrogolglycerol hydroxystearate is a mixture of approximately 75% relatively hydrophobic of which a major portion is glycerol polyethylene glycol 12-oxystearate.

When preparing a formulation of the extracted xanthohumol/curcumin and non-ionic surfactant, typically, these two ingredients can be present at a weight ratio is from about 1:5 to about 1:200, though ratios outside of this range can also be used. Thus, in the water-soluble concentrate or gel, these two ingredients may be the only two ingredients present, and they can be present within this ratio range. If admixed with a solid of liquid carrier and/or other excipients, this ratio can remain the same, such as when water is added to form a liquid beverage, or a solid carrier is added to form a tablet or capsule or food supplement.

For the preparation of consumable formulations from the pharmaceutical formulations, pharmaceutically acceptable carriers can be either solid or liquid. Solid form preparations include powders, tablets, bilayer tablets or capsules, pills, capsules, cachets, suppositories, and dispersible granules. A solid carrier can be one or more substances, which may also act as diluents, flavoring agents, binders, preservatives, tablet disintegrating agents, or an encapsulating material. Details on techniques for formulation and administration are well described in the scientific and patent literature, see, e.g., the latest edition of Remington's Pharmaceutical Sciences, Maack Publishing Co, Easton Pa.

Suitable carriers include magnesium carbonate, magnesium stearate, talc, sugar, lactose, pectin, dextrin, starch (from corn, wheat, rice, potato, or other plants), gelatin, tragacanth, a low melting wax, cocoa butter, sucrose, mannitol, sorbitol, cellulose (such as methyl cellulose, hydroxypropylmethyl-cellulose, or sodium carboxymethylcellulose), and gums (including arabic and tragacanth), as well as proteins such as gelatin and collagen. If desired, disintegrating or co-solubilizing agents may be added, such as the cross-linked polyvinyl pyrrolidone, agar, alginic acid, or a salt thereof, such as sodium alginate. In powders, the carrier is a finely divided solid, which is in a mixture with the finely divided active component. In tablets, the active component is mixed with the carrier having the necessary binding properties in suitable proportions and compacted in the shape and size desired.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (i.e., dosage). Pharmaceutical preparations of the invention can also be used orally using, for example, push-fit capsules made of gelatin, as well as soft, sealed capsules made of gelatin and a coating such as glycerol or sorbitol. Push-fit capsules can contain xanthohumol and curcumin mixed with a filler or binders such as lactose or starches, lubricants such as talc or magnesium stearate, and, optionally, stabilizers. In soft capsules, the xanthohumol/curcumin compounds may be dissolved or suspended in suitable liquids, such as fatty oils, surfactants, non-ionic surfactants, liquid paraffin, or liquid polyethylene glycol with or without stabilizers.

After addition of the carrier to the pharmaceutical formulation, the consumable formulation may take the form of a water-soluble formulation, a beverage formulation, a food, formulation, a capsule formulation, a tablet formulation, an injectable formulation, a transdermal formulation, and any combination thereof.

In some embodiments, the water soluble formulation for administration can be a water solubilized formulation. A “water solubilized formulation,” as used herein, includes a prenylflavonoid such as xanthohumol, a polyphenol such as curcumin, and a non-ionic surfactant, and water (e.g. a water containing liquid) but does not include organic solvents (e.g. ethanol). In some embodiments, the water solubilized formulation a transparent water soluble formulation.

In one aspect, the present disclosure provides a water-soluble formulation for administration that comprises or consists essentially of a prenylflavonoid, such as xanthohumol, the polyphenol curcumin, and a non-ionic surfactant, and optionally water and/or excipients. In some embodiments, the water soluble formulation does not include a vegetable oil suspension or visible macro-micelles (micelles visible to the naked eye) in water. In other embodiments, the water soluble formulation does not include an alcohol (e.g. the compound is not first dissolved in alcohol and then added to water). In another aspect, the free form of the compound is preferred due to a higher concentration of the active compound.

In some embodiments, the water soluble formulation for administration includes the prenylflavonoid compound, or xanthohumol, the polyphenol compound curcumin, and polyoxyl castor oil to form a transparent water soluble formulation. In certain embodiments, light may be transmitted through the transparent water soluble formulations without diffusion or scattering. Thus, in some embodiments, the transparent water soluble formulations are not opaque, cloudy or milky-white. Transparent water soluble formulations disclosed herein do not include milky-white emulsions or suspensions in vegetable oil such as corn oil. Transparent water soluble formulations are also typically not formed by first dissolving the compound in alcohol, and then mixed with water.

In some embodiments, a water soluble formulation for administration can be in the form of a pharmaceutical composition or beverage. The pharmaceutical composition may include a prenylflavonoid such as xanthohumol, extracted from hops, the purified polyphenol curcumin, extracted from turmeric, and a non-ionic surfactant, and a pharmaceutically acceptable excipient, or water. After a pharmaceutical composition including the two ingredients of the invention has been formulated in an acceptable carrier, it can be placed in an appropriate container and labeled for treatment of an indicated condition. For administration of the two compounds, such labeling would include, e.g., instructions concerning the amount, frequency and method of administration.

In such embodiments, at least 0.5 mg, 1 mg, 2 mg, 3 mg, 4 mg, 5 mg, 10 mg, 20 mg, 30 mg, 40 mg, 50 mg, 100 mg, 200 mg, 300 mg, 400 mg, 500 mg, or 1 g of xanthohumol is present in the water soluble formulation. In other embodiments, 0.1 mg to 2 g, 0.5 mg to 1 g, 1 mg to 500 mg, 1 mg to 100 mg, 1 mg to 50 mg, 1 mg to 10 mg, or 1 mg to 5 mg of xanthohumol is present in the water soluble formulation.

In such embodiments, at least 0.5 mg, 1 mg, 2 mg, 3 mg, 4 mg, 5 mg, 10 mg, 20 mg, 30 mg, 40 mg, 50 mg, 100 mg, 200 mg, 300 mg, 400 mg, 500 mg, or 1 g of curcumin is present in the water soluble formulation. In other embodiments, 0.1 mg to 2 g, 0.5 mg to 1 g, 1 mg to 500 mg, 1 mg to 100 mg, 1 mg to 50 mg, 1 mg to 10 mg, or 1 mg to 5 mg of curcumin is present in the water soluble formulation.

Any appropriate dosage form is useful for administration of the water soluble formulation of the present invention, such as oral, parenteral and topical dosage forms. Oral preparations include tablets, pills, powder, dragees, capsules (e.g. soft-gel capsules), liquids, lozenges, gels, syrups, slurries, beverages, suspensions, etc., suitable for ingestion by the patient. The formulations of the present invention can also be administered by injection, that is, intravenously, intramuscularly, intracutaneously, subcutaneously, intraduodenally, or intraperitoneally. Additionally, the formulations of the present invention can be administered transdermally. The formulations can also be administered by in intraocular, intravaginal, and intrarectal routes including suppositories. Thus, the formulations described herein may be adapted for oral administration.

In dietary compositions for administration, especially in food and beverages for humans, the prenylflavonoid or xanthohumol or any mixture of them, and turmeric extract or curcumin is suitably present in an amount in the range of from about 0.0001 (1 mg/kg) to about 5 weight-% (50 g/kg), preferably from about 0.001% (10 mg/kg) to about 1 weight-%, (10 g/kg) more preferably from about 0.01 (100 mg/kg) to about 0.5 weight-% (5 g/kg), based upon the total weight of the food or beverage. Beverages encompass non-alcoholic and alcoholic drinks as well as liquid preparations to be added to drinking water and liquid food. Non-alcoholic drinks are e.g. soft drinks, sport drinks, fruit juices, lemonades, near-water drinks (i.e. water-based drinks with low calorie content), teas and milk-based drinks. Liquid foods are e.g. soups and dairy products.

Xanthohumol may also be present, for example, in a tablet formulation, at a concentration from 0.5 to 50 mg per tablet combined with Curcumin at a concentration of 1 to 1,000 mg. In other embodiments, the xanthohumol is present at a concentration from 0.01 mg/ml to 25 mg/tablet, and the curcumin from 25-500 mg. per tablet or capsule. The formulations may be administered as a unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The quantity of active component in a unit dose preparation may be varied or adjusted according to the particular application and the potency of the active component. The composition can, if desired, also contain other compatible therapeutic agents.

As mentioned, when preparing a suitable formulation, any formulation that effectively delivers the combination of xanthohumol and curcumin can be prepared. In one specific example, the xanthohumol can be prepared in a formulation by heating a water-soluble non-ionic surfactant in a container to a temperature of about 90° F. to about 200° F. while mixing the non-ionic surfactant until a clear non-ionic surfactant is formed; and adding the xanthohumol or curcumin to the clear non-ionic surfactant and mixing until a clear non-ionic surfactant-xanthohumol/curcumin combination is formed so as to constitute from about 4 wt % to 40 wt % surfactant and from 0.01 wt % to 10 wt % xanthohumol or curcumin. The xanthohumol/curcumin is thus sufficiently dispersed or dissolved in the surfactant so that a gel composition or emulsion is formed containing no visible micelles or particles of xanthohumol or curcumin. The solution may be heated to increase solubility. The heating temperature is typically selected to avoid chemical breakdown of the non-ionic surfactant. Likewise, the curcumin component can be prepared separately in the same manner. Water can be used to solvate the gel or concentrate of each emulsion to make a beverage in one example, or can be fortified directly (with or without added water) into soft gel capsules, creams, ointments, foods, etc.

In one specific embodiment, the temperature of both can be maintained at from 90 and 150° F. In some embodiments, the resulting solution is a water-soluble formulation or transparent water soluble formulation as described above. For example, the resulting solution may be a water soluble formulation that is a crystal clear solution, with no particles visible to the naked eye. Alternatively, the gel composition (prior to addition with water) will be combinable with warm water, as described above, to form a water soluble formulation.

In another aspect, the disclosure relates to the use of an effective amount of a prenylflavonoid, e.g., xanthohumol, or any mixture thereof, and an extract of turmeric containing curcumin for the manufacture of a composition for the treatment of a disorder connected to mood or stress, such as depression, dysthymia, generalized anxiety disorder, cognitive dysfunction, impaired memory, mental fatigue, physical fatigue, emotional imbalance, occupational stress, insomnia, dysregulation of Circadian rhythm, and antisocial behavior. The invention relates to the use of an effective amount of a prenylflavonoid, such as xanthohumol, or any mixture thereof, and an extract of turmeric containing curcumin, particularly for the manufacture of an antidepressant, a mood/vitality improver, a stress reliever, a condition improver, a reducer of anxiety, a reducer of obsessive-compulsive behavior, a relaxant, a sleep improver and/or an insomnia alleviator and a cognitive enhancer. The disclosure also relates to the use of an effective amount of hops xanthohumol, or any mixture thereof, combined with turmeric or curcumin, formulated with a non-ionic surfactant for the manufacture of a water soluble, or gastric soluble, composition for the treatment of a disorder connected to mood or stress, particularly for the manufacture of an antidepressant, a mood/vitality improver, a stress reliever, a condition improver, a reducer of anxiety, a reducer of obsessive-compulsive behavior, a relaxant, a sleep improver and/or an insomnia alleviator and a cognitive enhancer. Thus, the present invention is also directed to a method for the prevention of a mood disorder in animals including humans, said method comprising administering an effective dose of a prenylflavonoid, such as xanthohumol or any mixture thereof to animals including humans which are in need thereof. In this regard an effective dose of curcumin and xanthohumol, may especially be used for maintaining the mental well-being, for maintaining a balanced cognitive function, for helping to reduce the risk of mood swings, for helping to retain a positive mood and for supporting cognitive wellness, and for helping to maintain a good sleep quality.

In another aspect, the disclosure relates to the use of an effective amount of, xanthohumol, combined with turmeric or curcumin, or any mixture thereof, for the manufacture of a composition for enhanced activity or increase in the half-life or duration of activity of curcumin in the blood stream.

The amount of xanthohumol sufficient to have a therapeutic effect on a subject with an affective mood disorder condition may be from about 0.5 mg to about 1000 mg, from about 1 mg to about 50 mg, from about 1 mg to about 20 mg, or about 3 mg to about 10 mg. In some embodiments, the dose of xanthohumol is 1 mg, 3 mg, 5 mg, 10 mg, or 20 mg. or 50 mg. In still other embodiments, the dose of xanthohumol is about 5 mg. The xanthohumol is typically administered as a twice per day formulation or as a once per day formulation.

The amount of curcumin sufficient to have a therapeutic effect on a subject with an affective mood disorder condition may be from about 25 mg to about 1000 mg, from about 50 mg to about 500 mg, from about 1 mg to about 20 mg, or about 3 mg to about 10 mg. if in a water-soluble formulation, or an emulsion with enhanced bioavailability. In some embodiments, the dose of curcumin is 5 mg, 10 mg, 15 mg, 20 mg, or 25 mg. or 50 mg. In still other embodiments, the dose of curcumin is about 50 mg. The curcumin is typically administered as a twice per day formulation or as a once per day formulation, and may be used in a beverage if in a water soluble formulation.

In solid dosage unit preparations for humans, xanthohumol and curcumin or any mixture of them, is suitably present in an amount in the range of from about 0.1 mg to about 1000 mg, preferably in the range of from about 1 mg to about 500 mg per dosage unit. More preferably, in a range of about 1 mg to about 100 mg. Dosages within these ranges can be relevant to both consumable compositions, as well as injectable or topical formulations, and can be modified within appropriate ranges as would be appreciated by one skilled in the art after considering the present disclosure.

In dietary compositions, especially in food and beverages for humans, the hops xanthohumol is suitably present in an amount in the range of from about 0.0001 (1 mg/kg) to about 5 weight-% (50 g/kg), preferably from about 0.001% (10 mg/kg) to about 1 weight %, (10 g/kg) more preferably from about 0.01 (100 mg/kg) to about 0.5 weight-% (5 g/kg), based upon the total weight of the food or beverage. In food and drinks, the range is from 5 to 50 mg per serving, i.e. about 120 mg per kg food or drink. The amount of turmeric extract or curcumin in an amount in the range of from about 0.0001 (1 mg/kg) to about 5 weight-% (50 g/kg), preferably from about 0.001% (10 mg/kg) to about 1 weight-%, (10 g/kg) more preferably from about 0.01 (100 mg/kg) to about 0.5 weight-% (5 g/kg), based upon the total weight of the food or beverage. In food and drinks, the range is from 5 to 500 mg per serving.

For animals excluding humans a suitable daily dosage of xanthohumol, may be within the range of from 0.001 mg per kg body weight to about 1000 mg per kg body weight per day. More preferred is a daily dosage in the range of from about 0.1 mg to about 500 mg per kg body weight, and especially preferred is a daily dosage in the range of from about 1 mg to 50 mg per kg body weight. A suitable daily dosage of turmeric or curcumin, or any mixture of them, may be within the range of from 0.001 mg per kg body weight to about 100 mg per kg body weight per day. More preferred is a daily dosage in the range of from about 0.1 mg to about 50 mg per kg body weight, and especially preferred is a daily dosage in the range of from about 1 mg to 25 mg per kg body weight. A preferred serving size of a suitable beverage, capsule, or tablet would contain a minimum of about 10 mg. xanthohumol and 50 mg. curcumin per serving.

A combination of xanthohumol and curcumin, may be incorporated into various types of dosage forms for convenient consumption by animals or humans. Examples of fortified foods are cereal bars and bakery items such as cakes and cookies. Additionally, the combination can be administered in the form of a beverage, food, feed, dairy product, yoghurt, fortified food, enhanced water, cereal bars, bakery item, cake, cookies, dietary supplement, tablet, pill, granules, dragees, capsules, effervescent formulations, non-alcoholic drinks, soft drinks, sport drinks, fruit juices, teas, milk-based drinks, liquid foods, soups, liquid dairy products, or any combination thereof.

Another embodiment can include observing a behavioral or autonomic effect of the combination on the patient and further modifying treatment based on the observed effect. Observed effects can include improved mood/vitality, relief of stress, reduced anxiety, reduced obsessive-compulsive behavior, relaxation, improved sleep, alleviated insomnia, enhanced of cognition, maintenance of cognitive wellness and balance, enhanced learning, enhanced language processing, enhanced problem solving, enhanced intellectual functioning, enhanced ability to cope with psychosocial burdens, enhanced attention and concentration, enhanced memory, enhanced mental alertness, enhanced mental vigilance, and stabilized mental status. In an embodiment using the combination for the treatment of skin inflammation, observed effect can include improved pruritis, decreased skin inflammation, and decreased blood levels of inflammatory cytokines.

The foregoing detailed description describes the disclosure with reference to specific exemplary embodiments. However, it will be appreciated that various modifications and changes can be made without departing from the scope of the present invention as set forth in the appended claims. The detailed description and accompanying drawings are to be regarded as merely illustrative, rather than as restrictive, and all such modifications or changes, if any, are intended to fall within the scope of the present invention as described and set forth herein.

### EXAMPLES

The xanthohumol used in the following Examples was extracted from hops flowers and was analyzed by HPLC. The curcumin was extracted from turmeric and was analyzed by HPLC.

### Example 1

Water soluble compositions of xanthohumol were formulated containing polyoxyl castor oil. The polyoxyl castor oil (non-ionic surfactant) was heated and stirred to a temperature of about 150° F., then, the powdered xanthohumol (a hops extract containing 42% xanthohumol) was added slowly and mixed until a clear viscous solution was formed containing dissolved xanthohumol (hereinafter referred to as “the emulsion phase,” “gel,” or “water-soluble concentrate”). The xanthohumol/surfactant mixture was then slowly added to warm water (120° F.) until a crystal clear solution was formed. The resulting concentration of xanthohumol in the water-soluble beverage base was 5 mg/ml. (Table 1).

Likewise, water soluble compositions of curcumin were formulated containing polyoxyl castor oil. The polyoxyl castor oil (non-ionic surfactant) was heated and stirred to a temperature of about 150° F., then, powdered curcumin (a turmeric extract containing 98% curcumin) was added slowly and mixed until a clear viscous solution was formed containing 5 wt % dissolved curcumin (hereinafter referred to as “the emulsion phase,” “gel,” or “water-soluble concentrate”). The curcumin/surfactant mixture was then slowly added to warm water (120° F.) until a crystal clear red solution was formed. The resulting water soluble beverage base contained a concentration of curcumin of about 2.5 mg/ml. (Table 2).

TABLE 1

| Ingredient | V % |
|---|---|
| Xanthohumol Extract (42%) | 1.3 |
| Water | 78.20% |
| Polyoxyl Castor Oil | 20% |
| Sodium Benzoate | 0.06% |
| Potassium Sorbate | 0.04% |
| Citric Acid | 0.4% |
| Total | 100% |

TABLE 2

| Ingredient | V % |
|---|---|
| Curcumin | 0.25% |
| Water | 94.5% |
| Polyoxyl Castor Oil | 4.75% |
| Sodium Benzoate | 0.06% |
| Potassium Sorbate | 0.04% |
| Citric Acid | 0.4% |
| Total | 100% |

The concentration of xanthohumol in Table 1 was tested by HPLC and contained 5 mg/ml xanthohumol. The concentration of curcumin in Table 2 was tested by HPLC and found to contain 2.5 mg/ml curcumin.

### Example 2

50 ml of the water soluble liquid concentrate of curcumin and 2 ml of the water-soluble liquid concentrate of xanthohumol from Example 1 was added to 500 ml of water to make a pleasant tasting beverage drink or fortified water. The beverage therefore delivered 10 mg. of xanthohumol and 125 mg. of curcumin per serving. 6 subjects suffering from mild depression and stress were instructed to consume a 500 ml bottle of this beverage once per day in the morning for two weeks, and were asked to fill out a daily questionnaire related to subjective feelings of improvement in mood, focus, emotional balance. Four of the six subjects experienced a significant improvement in mood and focus after 3 days of consuming the beverage. Two of the six subjects experienced a mild improvement, which varied from day to day, with some days experiencing a greater improvement than others.

### Example 3

In an effort to understand the activity of xanthohumol on the metabolism of curcumin the following study was conducted;

### Objective

The objective of this study was to determine the stability of curcumin in human liver microsomes in the absence and presence of xanthohumol at a concentration of 1 μM.

### Experimental Procedure

Mixed-gender human liver microsomes (Lot #1210347) were purchased from XenoTech. The reaction mixture, minus cofactors, was prepared as described below. The test article was added into the reaction mixture at a final concentration of 1 μM. second experiment was also performed with 1 μM xanthohumol also present in the reaction mixture. The control compounds, testosterone and 7-hydroxycoumarin (7-HC), were run simultaneously with the test article in separate reactions. An aliquot of the reaction mixture (without cofactors) was equilibrated in a shaking water bath at 37° C. for 3 minutes. The reaction was initiated by the addition of cofactors, and the mixture was incubated in a shaking water bath at 37° C. Aliquots (100 μL) were withdrawn at 0, 10, 20, 30, and 60 minutes for the test article and 0, 10, 30, and 60 minutes for testosterone and 7-HC. All samples were immediately combined with 400 μL of ice-cold 50/50 acetonitrile/H₂O containing 0.1% formic acid and internal standard to terminate the reaction. The samples were then mixed and centrifuged to precipitate proteins. All samples were assayed by LC-MS/MS using electrospray ionization. Analytical conditions are outlined in Appendix 1. The peak area response ratio (PARR) to internal standard was compared to the PARR at time 0 to determine the percent remaining at each time point. Half-lives were calculated using GraphPad software, fitting to a single-phase exponential decay equation.

### Reaction Composition

| | | |
|---|---|---|
| Liver Microsomes | 0.5 | mg/mL |
| NADPH (cofactor) | 1 | mM |
| UDPGA (cofactor) | 1 | mM |
| Potassium Phosphate, pH 7.4 | 100 | mM |
| Magnesium Chloride | 5 | mM |
| Test Article | 1 | μM |

### Experimental Results

| | % Remaining of Initial (n = 1) | | | | | | |
|---|---|---|---|---|---|---|---|
| | 0 | 10 | 20 | 30 | 60 | Half-life | CLᵢₙₜ (mL/min/ |
| Test Article | min | min | min | min | min | (min) | mg protein) |
| Curcumin | 100 | 20 | 6.9 | 2.8 | 0.5 | 4.4 | 0.313 |
| Curcumin + | 100 | 29 | 14 | 7.0 | 1.6 | 6.2 | 0.224 |
| 1 μM | | | | | | | |
| xanthohumol | | | | | | | |

| | | CLᵢₙₜ | Acceptable |
|---|---|---|---|
| Control | Half-life | (ml/min/mg | Range |
| Compound | (min) | protein) | (t_{1/2}, min) |
| Testosterone | 19 | 0.0740 | ≦41 |
| 7-HC | 10 | 0.136 | N/A |

N/A: not applicable

### Analytical Method

Liquid Chromatography

Column: Thermo BDS Hypersil C18 30×2.0 mm, 3 μm, with guard column

M.P. Buffer: 25 mM ammonium formate buffer, pH 3.5

Aqueous Reservoir (A): 90% water, 10% buffer

Organic Reservoir (B): 90% acetonitrile, 10% buffer

Flow Rate: 350 μL/minute

Gradient Program:

| Time (min) | % A | % B |
|---|---|---|
| 0.0 | 100 | 0 |
| 0.5 | 50 | 50 |
| 1.0 | 0 | 100 |
| 1.5 | 0 | 100 |
| 1.6 | 100 | 0 |
| 2.5 | 100 | 0 |

Total Run Time: 2.5 minutes

Autosampler: 5 μL Injection Volume

Autosampler Wash: water/methanol/2-propanol:1/1/1; with 0.2% formic acid

Mass Spectrometer

Instrument: PE SCIEX API 4000

Interface: Turbo Ionspray

Mode: Multiple reaction monitoring

Method: 2.5 minute duration

Settings:

| Test Article | Q1/Q3 | DP | EP | CE | CXP | IS | TEM | CAD | CUR | GS1 | GS2 |
|---|---|---|---|---|---|---|---|---|---|---|---|
| Curcumin | +369.2/177.1 | 70 | 10 | 29 | 10 | 5500 | 500 | 7 | 20 | 20 | 30 |

### Example 4

The objective of this study was to determine the stability of curcumin in human liver microsomes in the absence and presence of xanthohumol at a concentration of 10 μM, which is 10 fold higher than the previous study.

### Summary

| | | CLᵢₙₜ |
|---|---|---|
| Test Article | Half-life (min) | (mL/min/mg protein) |
| Curcumin | <10 (4.7) | >0.139 (0.296) |
| Curcumin + | 19 | 0.0711 |
| 10 μM xanthohumol | | |

### Objective

The objective of this study was to determine the stability of curcumin in human liver microsomes in the absence and presence of 10 μM xanthohumol.

### Experimental Procedure

Mixed-gender human liver microsomes (Lot #1210347) were purchased from XenoTech. The reaction mixture, minus cofactors, was prepared as described below. The test article was added into the reaction mixture at a final concentration of 1 μM. A second experiment was also performed with 10 μM xanthohumol also present in the reaction mixture. The control compounds, testosterone and 7-hydroxycoumarin (7-HC), were run simultaneously with the test article in separate reactions. An aliquot of the reaction mixture (without cofactors) was equilibrated in a shaking water bath at 37° C. for 3 minutes. The reaction was initiated by the addition of cofactors, and the mixture was incubated in a shaking water bath at 37° C. Aliquots (100 μL) were withdrawn at 0, 10, 20, 30, and 60 minutes for the test article and 0, 10, 30, and 60 minutes for testosterone and 7-HC. All samples were immediately combined with 400 μL of ice-cold 50/50 acetonitrile/H₂O containing 0.1% formic acid and internal standard to terminate the reaction. The samples were then mixed and centrifuged to precipitate proteins. All samples were assayed by LC-MS/MS using electrospray ionization. Analytical conditions are outlined in Appendix 1. The peak area response ratio (PARR) to internal standard was compared to the PARR at time 0 to determine the percent remaining at each time point. Half-lives were calculated using GraphPad software, fitting to a single-phase exponential decay equation.

### Reaction Composition

| | | |
|---|---|---|
| Liver Microsomes | 0.5 | mg/mL |
| NADPH (cofactor) | 1 | mM |
| UDPGA (cofactor) | 1 | mM |
| Potassium Phosphate, pH 7.4 | 100 | mM |
| Magnesium Chloride | 5 | mM |
| Test Article | 1 | μM |

### Experimental Results

| | % Remaining of Initial (n = 1) | | | | | | |
|---|---|---|---|---|---|---|---|
| | 0 | 10 | 20 | 30 | 60 | Half-life | CLᵢₙₜ (mL/min/ |
| Test Article | min | min | min | min | min | (min) | mg protein) |
| Curcumin | 100 | 22 | 6.6 | 2.8 | 0.5 | <10 (4.7) | >0.139 (0.296) |
| Curcumin + | 100 | 65 | 45 | 36 | 15 | 19 | 0.0711 |
| 10 μM | | | | | | | |
| xanthohumol | | | | | | | |

| | | CLᵢₙₜ | Acceptable |
|---|---|---|---|
| Control | Half-life | (ml/min/mg | Range |
| Compound | (min) | protein) | (t_{1/2}, min) |
| Testosterone | 24 | 0.0577 | ≦41 |
| 7-HC | 12 | 0.120 | N/A |

N/A: not applicable

### APPENDIX 1. ANALYTICAL METHOD

Liquid Chromatography

Column: Thermo BDS Hypersil C18 30×2.0 mm, 3 μm, with guard column

M.P. Buffer: 25 mM ammonium formate buffer, pH 3.5

Aqueous Reservoir (A): 90% water, 10% buffer

Organic Reservoir (B): 90% acetonitrile, 10% buffer

Flow Rate: 350 μL/minute

Gradient Program:

| Time (min) | % A | % B |
|---|---|---|
| 0.0 | 100 | 0 |
| 0.5 | 50 | 50 |
| 1.0 | 0 | 100 |
| 1.5 | 0 | 100 |
| 1.6 | 100 | 0 |
| 2.5 | 100 | 0 |

Total Run Time: 2.5 minutes

Autosampler: 5 μL Injection Volume

Autosampler Wash: water/methanol/2-propanol:1/1/1; with 0.2% formic acid

Mass Spectrometer

Instrument: PE SCIEX API 4000

Interface: Turbo Ionspray

Mode: Multiple reaction monitoring

Method: 2.5 minute duration

Settings:

| Test Article | Q1/Q3 | DP | EP | CE | CXP | IS | TEM | CAD | CUR | GS1 | GS2 |
|---|---|---|---|---|---|---|---|---|---|---|---|
| Curcumin | +369.2/177.1 | 70 | 10 | 29 | 10 | 5500 | 500 | 7 | 20 | 20 | 30 |

As can be seen from the above study, when xanthohumol is added curcumin, the metabolism of the curcumin is significantly improved. The half-life of curcumin in human liver microsomes is less than 10 minutes, but when xanthohumol is added to the curcumin, the half-life is almost doubled, to about 19 minutes.

What is claimed is:

1. A metabolic enhancing pharmaceutical composition comprising xanthohumol and curcumin.

2. The composition of claim 1, wherein the curcumin is from a curcumin extract and the xanthohumol is from a xanthohumol extract from hops.

3. The composition of claim 1, wherein the xanthohumol is present at a concentration from about 0.01% to 50% by weight, and the curcumin is present at a concentration of from 0.01% to 10% by weight.

4. The composition of claim 1, wherein the formulation further comprises an emulsifier, ethanol, d-alpha tocopheryl polyethylene glycol succinate, glycerin, cycoldextrin, phosphatidylcholine, PEGylated nanospheres, PLGA nanospheres, or nanoparticles.

5. The composition of claim 1, wherein the formulation further comprises a non-ionic surfactant.

6. The composition of claim 5, wherein the non-ionic surfactant is selected from the group comprising non-ionic water soluble mono-, di-, or tri-glycerides; non-ionic water soluble mono- or di-fatty acid esters of polyethylene glycol; non-ionic water soluble sorbitan fatty acid esters; polyglycolyzed glycerides; non-ionic water soluble triblock copolymers; their derivatives; and combinations thereof.

7. The composition of claim 5, wherein the non-ionic surfactant is a non-ionic water-soluble mono-, di-, or tri-glyceride.

8. The composition of claim 5, wherein the non-ionic surfactant is polyoxyl castor oil.

9. The composition of claim 5, wherein the xanthohumol and curcumin are present in an extract, and the extract to non-ionic surfactant weight ratio is from about 1:5 to about 1:200.

10. A method of enhancing the metabolic activity or stability of a curcumin extract, comprising preparing a formulation by combining the curcumin extract with an extract of xanthohumol from hops.

11. The method of claim 10, wherein the curcumin and xanthohumol are formulated with a solubility enhancing agent.

12. The method of claim 11, wherein the solubility enhancing agent is an emulsifier, ethanol, non-ionic surfactant, d-alpha tocopheryl polyethylene glycol succinate, glycerin, cycoldextrin, phosphatidylcholine, PEGylated nanospheres, PLGA nanospheres, or nanoparticles.

13. The method of claim 10, further comprising administering the formulation to a subject which experiences enhanced metabolic stability compared to the curcumin extract administered without the extract of xanthohumol from hops.

14. The method of claim 10, wherein the formulation is in the form of a water soluble gel or concentrate.

15. The method of claim 10, wherein the formulation further comprises a solid or liquid carrier suitable to form a consumable formulation.

16. The method of claim 15, wherein the solid or liquid carrier is the solid carrier, and is selected from a group consisting of magnesium carbonate, magnesium stearate, talc, sugar, lactose, pectin, dextrin, starch, gelatin, tragacanth, a low melting wax, cocoa butter, vegetable oil, ethanol, sucrose, mannitol, sorbitol, cellulose, gums, gelatin, collagen, and combinations thereof.

17. The method of claim 15, wherein the solid or liquid carrier is the liquid carrier, and the consumable formulation is a beverage formulation.

18. The method of claim 10, wherein the extract of xanthohumol and curcumin extract are administered as a consumable formulation selected from a group comprising beverages, food, feed, dairy products, yoghurts, fortified food, enhanced waters, cereal bars, bakery items, cakes, cookies, dietary supplements, tablets, pills, granules, dragees, capsules, effervescent formulations, non-alcoholic or alcoholic drinks, soft drinks, sport drinks, fruit juices, teas, milk-based drinks, liquid foods, soups, liquid dairy products, and any combination thereof.
